(19) United States
(12) Patent Application Publication
(10) Pub. No.: US 2024/0252208 A1
(43) Pub. Date: Aug. 1, 2024

(54) LEAD RETAINING DEVICE

(71) Applicant: XTRAC O.S LTD, Raanana (IL)

(72) Inventor: Uri STEIN, Ramat Gan (IL)

(73) Assignee: XTRAC O.S LTD, Raanana (IL)

(21) Appl. No.: 18/564,490

(22) PCT Filed: May 29, 2022
(86) PCT No.: PCT/IL2022/050571

§ 371 Date: Nov. 27, 2023

(30) Foreign Application Priority Data
May 30, 2021 (IL) 283558

(51) Int. Cl. A61B 17/34

(52) U.S. Cl. CPC A61B 17/3468

(57) ABSTRACT

The present disclosure provides a device for assisting in the extraction of a lead of a pacemaker or implantable cardioverter defibrillator (ICD) from a subject. The device is therefore capable of correcting most life-threatening cardiac arrhythmias. The device is designed to prevent any movement of the lead and/or the coil of the pacemaker or ICD and allowing to extract the lead by an anchoring member (e.g. a stylet that is designed to be introduced into that lead and to anchor in a certain portion within the lumen of the lead) and/or applying vibrations thereto through the anchoring member. The device is configured to allow the anchoring member to pass therethrough such that it is locked to a certain position within the lumen of the lead at a first end thereof and the second end is available for applying vibrations thereto and pulling it to extract the lead.

### TECHNOLOGICAL FIELD

The present disclosure is in the field of pacemaker's or implantable cardioverter defibrillator's lead extraction assisting devices.

### BACKGROUND ART

References considered to be relevant as background to the presently disclosed subject matter are listed below:
WO 2019/111255
U.S. Pat. No. 8,532,792
U.S. Pat. No. 5,697,804

Acknowledgement of the above references herein is not to be inferred as meaning that these are in any way relevant to the patentability of the presently disclosed subject matter.

### GENERAL DESCRIPTION

The present disclosure provides a device for assisting in the extraction of a lead of a pacemaker or implantable cardioverter defibrillator (ICD) from a subject. It is to be noted that the terms “pacemaker” and ICD are interchangeable throughout the description, and they should be inferred as any device that is implantable inside the body, able to perform cardioversion, defibrillation, or pacing of the heart. The device is therefore capable of correcting most life-threatening cardiac arrhythmias. The device is designed to prevent any movement of the lead and/or the coil of the pacemaker or ICD and allowing to extract the lead by an anchoring member (e.g. a stylet that is designed to be introduced into that lead and to anchor in a certain portion within the lumen of the lead) and/or applying vibrations thereto through the anchoring member. The device is configured to allow the anchoring member to pass therethrough such that it is locked to a certain position within the lumen of the lead at a first end thereof and the second end is available for applying vibrations thereto and pulling it to extract the lead.

Therefore, an aspect of the present disclosure provides a device for retaining a coil element of a lead of a pacemaker or ICD for allowing its extraction from the body it is implanted in. The device includes a lead receiving element that is formed with a lumen extending along an axis between a proximal opening and a distal opening of said lead receiving element. At least the proximal opening is shaped and sized to receive said lead and at least a portion of the lumen that is at the vicinity of the proximal opening is also shaped and sized to receive said lead.

A coil engaging element is defined between a first engaging end and a second end. The coil engaging element is fixed with respect to the lumen, namely it can be fixed indirectly, e.g., via another element, or directly to the lead receiving element such that it is remains in a constant relation to the lumen or a portion of it. The first end of the coil engaging element is configured to engage a coil of a lead of a pacemaker or ICD that is disposed within the lumen of the lead. The engagement of the coil engaging element is intended to engage the end of said coil and to compress it upon application of force along a common axis defined by the lumen and the coil engaging element. The coil engaging element is formed with a tunnel between the first end and the second end and the tunnel is shaped and sized for allowing passing of a lead anchoring element/stylet or part of it therethrough, e.g., a thread of a lead anchoring element.

The device further includes a retaining mechanism that is configured to selectively switch between (i) a retaining state, in which it retains said lead in position with respect to the coil engaging element, and (ii) a non-retaining state, in which it allows movement of said lead with respect to the coil engaging element within said lumen. When in the retaining state, typically the lead is retained within said lumen when the lead is received within the lumen. It is to be noted that the coil engaging element may affect the movement of the lead as it engages the coil of the lead and, therefore, may resist some of the movement of the lead, even if the retaining mechanism is in the non-retaining state.

Yet another aspect of the present disclosure provides a device for retaining a lead of a pacemaker or ICD for allowing its extraction from the body. The device comprising a lead receiving element having a lumen extending along an axis between a proximal opening and a distal opening of said lead receiving element. The proximal opening is shaped and sized to receive said lead, and said distal opening is shaped and sized for (i) blocking passing of the lead and (ii) allowing passing of a lead anchoring element/stylet or part of it therethrough, e.g., a thread of a lead anchoring element. It is to be understood that the thread is a part of the stylet that is introduced into the lead and is configured to be anchored at a desired location within the lumen of the coil that is disposed within the lumen of the lead.

At least a portion of the lead receiving element is deformable upon external application of force thereon resulting in at least one of: (i) a reduction of at least a portion of the lumen cross section or (ii) an increase of pressure on a lead received in the lumen, thereby constituting a retaining mechanism configured to selectively switch between (i) a retaining state, in which it retains said lead in position within the lumen, and (ii) a non-retaining state, in which it allows movement of said lead within the lumen. In other words, the degree of deformation of the lead receiving element correlates with a degree of pressure applied on a lead that is received in the lumen of the lead receiving element, such that sufficiently high-pressure results in the retention of the lead in the lumen and below this threshold the lead is free to move in the lumen.

The terms “deformable” or “deformation” should be interpreted as relating to a change in the structure of an element not limited to a certain change, for example, whether it is due to compression or expansion of the element or a movement of the element with respect to another element, e.g., a change of an angle of one part of the element with respect to another part, or any combination thereof.

The following embodiments with respect to the device are applied to any of the two aspects described above and any combination of embodiments can be obtained by the present disclosure.

In more general, it is to be noted that any combination of the described embodiments with respect to any aspect of this present disclosure is applicable. In other words, any aspect of the present disclosure can be defined by any combination of the described embodiments.

In some embodiments, the device comprising a coil engaging element defined between a first, engaging end and a second end, fixed with respect to the lumen. It is to be noted that the coil engaging element can be fixed indirectly or directly to the lead receiving element such that it is remained in a constant relation to the lumen or a portion of it. The first end is configured to engage a coil of said lead, wherein the coil engaging element is formed with a tunnel between the first end and the second end. The tunnel is shaped and sized for allowing passing of a thread linked, directly or indirectly to said lead. In some embodiments of the device, the retaining mechanism, or at least the active portion of the retaining mechanism that engages the lead, is within said lumen between the lead receiving element and the coil engaging element.

In some embodiments of the device, the first end of the coil engaging element extends over said proximal opening. In other words, the coil engaging element projects through the proximal opening such that a part of it is disposed within the lumen and a part of it is disposed outside the lumen.

In some embodiments of the device, the retaining mechanism comprises two or more clamping members that reside within the lumen and defining together a clamping state. The clamping state is defined by the dimension of the space or distance between the clamping members that defines whether it tightly embrace the lead or not. The clamping members are adjustable to allow a first range of clamping states defining said retaining state and a second range of clamping state defining said non-retaining state.

In some embodiments, the device further includes an adjuster configured to controllably adjust the clamping state of the clamping members. The adjuster may be disposed external to the lumen and the utilization thereof affects the clamping state within the lumen.

In some embodiments of the device, a portion of the coil engaging element extends in a space defined between the clamping members.

In some embodiments of the device, the coil engaging element extending along the entire lumen and projecting through said proximal and distal openings.

In some embodiments of the device, the tunnel and the lumen are coaxial and concentric.

In some embodiments, the device comprising a mounting member configured for mounting the coil engaging element within the lumen.

In some embodiments of the device, the mounting member is fixed to or integral with a part of the retaining mechanism.

In some embodiments of the device, the mounting member is disposed external to said lumen at a distal side. Namely, the mounting member is disposed further to the distal opening such that the coil engaging element extending from the mounting member, through the distal opening in the lumen and optionally through the proximal opening and further to the proximal side.

In some embodiments of the device, the clamping members extend from the mounting member or from a base structure that comprises said mounting member.

In some embodiments of the device, at least a portion of the lead receiving element is deformable to decrease the lumen dimension and/or allowing to embrace said lead when it is received in the lumen, thereby constituting the retaining mechanism.

In some embodiments of the device, the retaining mechanism is constituted by a deformation capability of at least a portion of the lead receiving element allowing to decrease the lumen volume and/or allowing to embrace said lead when it is received in the lumen.

In some embodiments of the device, the lead receiving element comprises or is formed of a pliable material allowing said deformation.

In some embodiments of the device, the retaining mechanism comprises an embracing member embracing at least a portion of said lead receiving element. The embracing member is capable of selectively adjustment along a range of embracing pressures on said lead receiving element, between a minimal embracing pressure and a maximal embracing pressure, thereby resulting in the switch between said non-retaining state and said retaining state. Namely, when the embracing member is adjusted to apply sufficiently high pressure on the lead receiving element, the lead receiving element is pressed on the lead that is received in its lumen and therefore the lead is tightly held in position within the lumen. The embracing member is formed with an embracing member proximal opening for allowing the passage of the lead therethrough, and therefore to be received in the lead receiving member, and an embracing member distal opening to allow the passage of the thread linked to the lead therethrough, typically after it exits the distal end of the lumen of the lead receiving element. The embracing member comprises an embracing member lumen extending between the proximal opening and the distal opening and the lead receiving element is disposed within said lumen and comes in contact with the walls of the lumen, at least upon deformation of the embracing member. In some embodiments of the device, the embracing member comprises an internal lumen and the lead receiving element is disposed within said internal lumen.

In some embodiments of the device, said embracing member is deformable so as to allow said adjustment along a range of embracing pressures.

In some embodiments of the device, the embracing member comprises an external thread on its external surface for allowing an internal thread of a deformation-inducing element to be threaded thereon, resulting in the deformation of the lead receiving element. Typically, the threading operation starts from the side of the embracing member distal opening towards the side of the embracing member proximal opening, and as the threading proceeds in this direction, the amount of pressure applied on a lead received in the lumen of the lead receiving element increases until it is sufficient to retain the lead in position.

The deformation-inducing element is a hollowed element that has an internal thread configured to be threaded to the external thread of the embracing member.

In some embodiments of the device, at least a portion of said embracing member has a reducing dimension profile of a cross section along a central axis thereof from a proximal end towards a distal end, and wherein the deformation-inducing element has a constant cross section dimension along a central axis thereof, thereby the position of the deformation-inducing element over the external surface defines the amount of pressure applied in retaining said lead.

In some embodiments of the device, the embracing member is formed with an external thread on its external surface configured to receive a respective internal threading of said deformation-inducing element. The device of any of the above-described embodiments may be used in a method for extracting a lead of a pacemaker or ICD from a subject.

Another aspect of the present disclosure provides a method for extracting a lead of a pacemaker or ICD. The method includes:
introducing an anchoring member, or a part thereof, into a central portion of a coil disposed in said lead, namely into a tunnel formed at the center of the coil;
passing the anchoring member, or an element coupled thereto, through a tunnel of a coil engaging element;
engaging the coil with said coil engaging element and compressing it to a desired extent;
retaining the lead in position with respect to the coil engaging element;
locking the anchoring member in a certain position within the lead; and
extracting the lead.

It is to be noted that the steps of the method are interchangeable, in particular the steps of introducing the anchoring member into the lead and passing the anchoring member through the tunnel.

Yet another aspect of the present disclosure provides a method for extracting a lead of a pacemaker or ICD. The method comprising:
introducing an anchoring member into a central lumen of a coil disposed in said lead;
anchoring the anchoring member in a certain position within the lead;
passing a thread of the anchoring member from a proximal end of a lumen of a lead receiving element to a distal end of said lumen receiving element;
receiving a portion of said lead in said lumen of the lead receiving element;
deforming said lead receiving element for retaining the lead in position with respect to the lumen; and
extracting the lead by applying extracting force profile on said thread.

It is to be noted that the steps of the method are interchangeable.

The following embodiments with respect to the method can be applied to any of the above-described method aspects and in any combination.

In some embodiments, the method comprising threading an internal thread of a deformation-inducing element over an external thread of said embracing member formed in its external surface, thereby resulting in said deforming and said retaining.

In some embodiments, the method further includes vibrating the locked anchoring member prior to said extracting.

In some embodiments, the above-described method is making use of a device of any of the above-described embodiments.

### EMBODIMENTS

The following are optional embodiments and combinations thereof in accordance with aspects of the present disclosure:
1. A device for retaining a lead of a pacemaker or ICD for allowing its extraction from the body, comprising:
   a lead receiving element having a lumen extending along an axis between a proximal opening and a distal opening of said lead receiving element, said proximal opening is shaped and sized to receive said lead, said distal opening is shaped and sized for (i) blocking passing of the lead and (ii) allowing passing of a part of a lead anchoring element anchored to said lead;
   wherein at least a portion of the lead receiving element is deformable upon external application of force thereon resulting in at least one of (1) a reduction of at least a portion of the lumen cross section or (2) an increase of pressure on a lead received in the lumen, thereby constituting a retaining mechanism configured to selectively switch between (i) a retaining state, in which it retains said lead in position within the lumen, and (ii) a non-retaining state, in which it allows movement of said lead within the lumen.
2. The device of embodiment 1, comprising a coil engaging element defined between a first end and a second end, fixed with respect to the lumen, the first end is configured to engage a coil of said lead, wherein the coil engaging element is formed with a tunnel between the first end and the second end, said tunnel is shaped and sized for allowing passing of a part of a lead anchoring element anchored to said lead.
3. A device for retaining a lead of a pacemaker or ICD for allowing its extraction from the body, comprising:
   a lead receiving element having a lumen extending along an axis between a proximal opening and a distal opening of said lead receiving element, said proximal opening is shaped and sized to receive said lead;
   a coil engaging element defined between a first end and a second end, fixed with respect to the lumen, the first end is configured to engage a coil of said lead, wherein the coil engaging element is formed with a tunnel between the first end and the second end, said tunnel is shaped and sized for allowing passing of a thread of a lead anchoring element therethrough;
   a retaining mechanism configured to selectively switch between (i) a retaining state, in which it retains said lead in position with respect to the coil engaging element, and (ii) a non-retaining state, in which it allows movement of said lead with respect to the coil engaging element.
4. The device of embodiment 3, wherein the retaining mechanism is within said lumen between the lead receiving element and the coil engaging element.
5. The device of embodiment 3 or 4, wherein the first end of the coil engaging element extends over said proximal opening.
6. The device of any one of embodiments 3-5, wherein the retaining mechanism comprises two or more clamping members that reside within the lumen and defining together a clamping state, the clamping members are adjustable to allow a first range of clamping states defining said retaining state and a second range of clamping state defining said non-retaining state.
7. The device of embodiment 6, comprising an adjuster configured to controllably adjust the clamping state of the clamping members.
8. The device of embodiment 6 or 7, wherein the coil engaging element extending in a space defined between the clamping members.
9. The device of any one of embodiments 2-8, wherein the coil engaging element extending along the entire lumen and projecting through said proximal and distal openings.
10. The device of any one of embodiments 2-9, wherein the tunnel and the lumen are coaxial.
11. The device of any one of embodiments 2-10, comprising a mounting member configured for mounting the coil engaging element within the lumen.
12. The device of embodiment 11, wherein the mounting member is fixed to or integral with a part of the retaining mechanism.
13. The device of embodiment 11 or 12, wherein the mounting member is disposed externally to said lumen at a distal side.
14. The device of any one of embodiments 1-13, wherein the retaining mechanism comprises two or more clamping members.
15. The device of any one of embodiments 2-13, comprising a mounting member configured for mounting the coil engaging element within the lumen, wherein said clamping members extend from the mounting member.
16. The device of embodiment 1-13, wherein at least a portion of the lead receiving element is deformable to decrease the lumen dimension and/or allowing to embrace said lead when it is received in the lumen, thereby constituting the retaining mechanism.
17. The device of embodiment 1-13, wherein the retaining mechanism is constituted by a deformation capability of at least a portion of the lead receiving element allowing to decrease the lumen volume and/or allowing to embrace said lead when it is received in the lumen.
18. The device of embodiment 16 or 17, wherein the lead receiving element comprises or is formed of a pliable material allowing said deformation.
19. The device of any one of embodiments 1-2, 16-18, wherein the retaining mechanism comprises an embracing member embracing at least a portion of said lead receiving element and is capable of selectively adjustment along a range of embracing pressures on said lead receiving element, between a minimal embracing pressure and a maximal embracing pressure, thereby resulting in the switch between said non-retaining state and said retaining state, wherein said embracing member is formed with an embracing member proximal opening for allowing the passage of the lead therethrough, and an embracing member distal opening to allow the passage of the thread linked to the lead therethrough.
20. The device of embodiment 19, wherein said embracing member is deformable so as to allow said adjustment along a range of embracing pressures.
21. The device of embodiment 20, wherein the embracing member is formed with an external thread on its external surface for allowing an internal thread of a deformation-inducing element to be threaded thereto, thereby resulting in the deformation of the lead receiving element.
22. The device of embodiment 20 or 21, wherein at least a portion of said embracing member has a reducing dimension profile of a cross section along a central axis thereof from a proximal end towards a distal end, and wherein the deformation-inducing element has a constant cross section dimension along a central axis thereof, thereby the position of the deformation-inducing element over the external surface defines the amount of pressure applied in retaining said lead.
23. The device of any one of embodiments 1-2 and 19-22, wherein the embracing member comprises an embracing member lumen extending between an embracing member proximal opening and an embracing member distal opening, wherein the lead receiving element is located within the embracing member lumen.
24. The device of embodiment 23, wherein the lead receiving element is in physical contact with the internal walls of the embracing member lumen.
25. A kit comprising the device of any one of embodiments 21-24 and said deformation-inducing element.
26. A method for extracting a lead of a pacemaker or ICD, comprising:
   introducing an anchoring member into a central lumen of a coil disposed in said lead;
   anchoring the anchoring member in a certain position within the lead;
   passing a gripping thread of the anchoring member through a tunnel of a coil engaging element;
   engaging the coil with said coil engaging element and compressing it to a desired extent;
   retaining the lead in position with respect to the coil engaging element; and
   extracting the lead by applying extracting force profile on said thread.
27. A method for extracting a lead of a pacemaker or ICD, comprising:
   introducing an anchoring member into a central lumen of a coil disposed in said lead;
   anchoring the anchoring member in a certain position within the lead;
   passing a thread of the anchoring member from a proximal end of a lumen of a lead receiving element to a distal end of said lumen receiving element;
   receiving a portion of said lead in said lumen of the lead receiving element;
   deforming said lead receiving element for retaining the lead in position with respect to the lumen; and
   extracting the lead by applying extracting force profile on said thread.
28. The method of embodiment 27, wherein said lead receiving element is embraced by an embracing member and the method comprising deforming at least a portion of said embracing member, resulting in said retaining.
29. The method of embodiment 28, wherein said deforming comprises threading an internal thread of a deformation-inducing element over an external thread of said embracing member, thereby resulting in said deforming and said retaining.
30. The method of any one of embodiments 26-29, comprising vibrating the locked anchoring member prior to said extracting.
31. A device according to any one of embodiments 1-24 for use in a method for extracting a lead of a pacemaker or ICD.
32. The device for use of embodiment 31, wherein the method is as defined in any one of embodiments 26-30.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1F are illustrations of different views of a non-limiting embodiment of the device for blocking a coil of a lead of a pacemaker or ICD according to an aspect of the present disclosure. FIG. 1A is a perspective view of the device; FIG. 1B is a longitudinal cross-section view of the device; FIG. 1C is a perspective view of the device; FIG. 1D is a side view of the device; FIG. 1E is a back view of the device; and FIG. 1F FIG. 1F is a cross-sectional view of the device and a part of a lead of a pacemaker or ICD to be inserted thereto and retained thereby.

FIGS. 2A-2B are flow diagrams exemplifying different embodiments of a method for extracting a lead of a pacemaker or ICD from a subject according to an aspect of the present disclosure.

FIGS. 3A-3C are schematic illustrations of different views of a non-limiting embodiment of an example of the device according to an aspect of the present disclosure. FIG. 3A is a perspective view; FIG. 3B is a cross-sectional view; and FIG. 3C is a cross-sectional view. FIGS. 3A-3B shows the device with the lead received therein and the anchoring element extending from the lead. FIG. 3C shows the device alone without the lead and the anchoring element.

### DETAILED DESCRIPTION

The following figures are provided to exemplify embodiments and realization of the invention of the present disclosure.

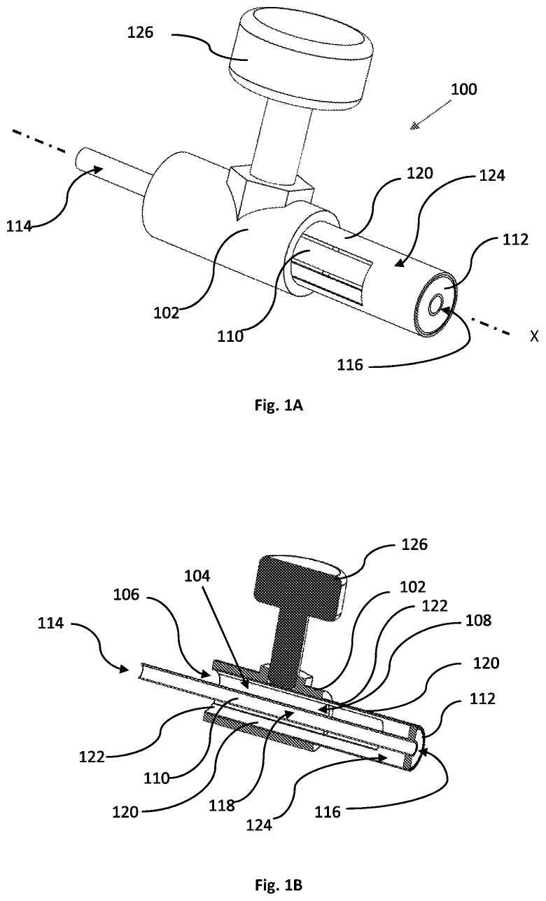

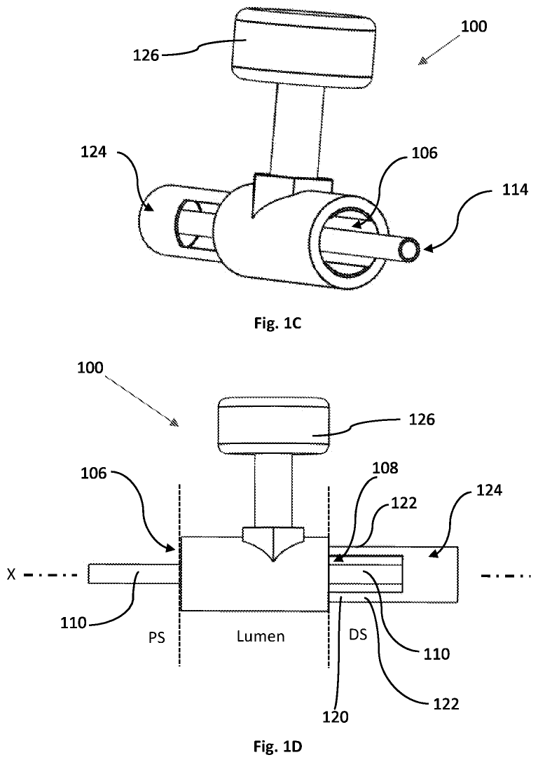

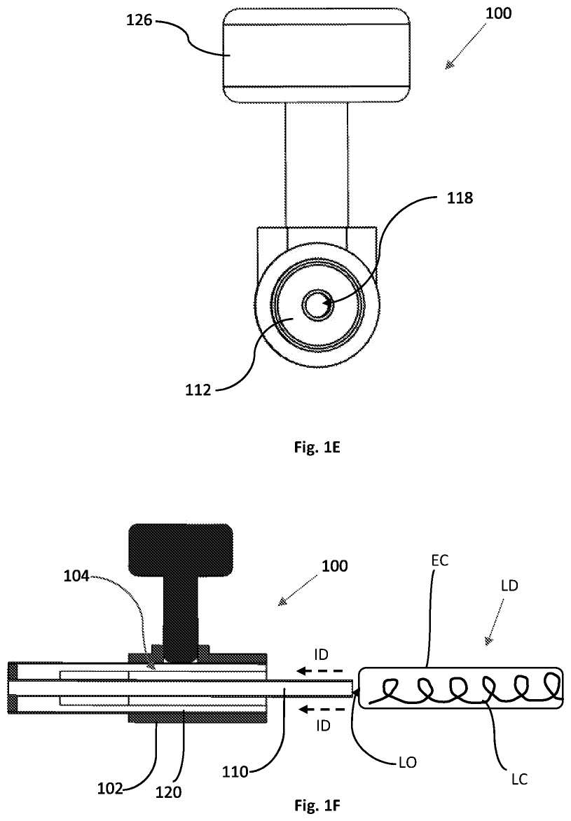

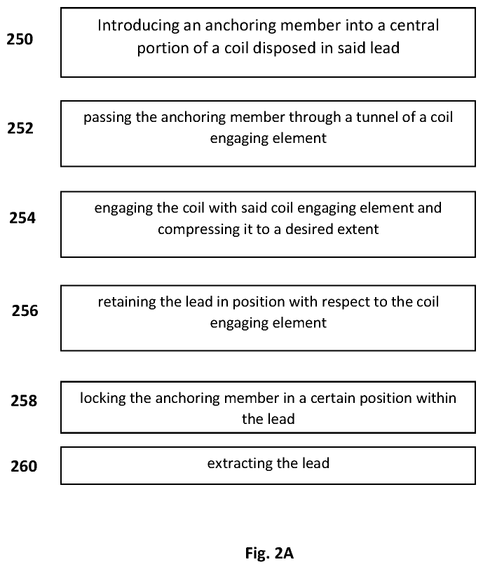

Reference is being made to FIGS. 1A-1F, which are illustrations of different views of a non-limiting embodiment of the device for blocking a coil of a lead of a pacemaker or ICD according to an aspect of the present disclosure. The device 100 includes a lead receiving element 102 that is hollowed and defining a lumen 104 between a proximal opening 106 and a distal opening 108, as can be best seen in FIG. 1B. The proximal side is the one that is intended to receive the lead and its coil. A coil engaging element 110, which is an elongated and hollowed element, is fixed to a mounting member 112 that is disposed distally to the distal opening 108 on a distal side DS of the lumen, such that the coil engaging element 110 extends from the proximal side PS to the distal side DS of the lumen 104, as can be best seen in FIG. 1D. In other words, the coil engaging element 110 is elongated to project from both, the proximal and the distal openings 106 and 108. The coil engaging element 110 includes a first, engaging end 114 that is configured to engage a coil of a lead to allow its compression to a certain desired extent, and an opposite, fixed end 116 that is fixed to the mounting member 112. The engaging end 114 is disposed at the proximal side PS of the lumen 104 and the fixed end 116 is disposed at the distal side DS of the lumen 104. The coil engaging element 110 is formed with a tunnel 118 that is designed to allow passing of an anchoring member, or a thread linked to the anchoring member, therethrough. The lumen 104 of the lead receiving element 102 and the tunnel 118 of the coil engaging element 110 are coaxial, therefore they are both extending along an axis X.

A retaining mechanism 120, which at least part thereof is disposed within the lumen 104, is configured to selectively switch between two states: (i) a first, retaining state that retains a lead when received within the lumen 104; and (ii) a second, non-retaining state that allows axial movement of the lead with respect to the coil engaging element 110 within the lumen 104. The retaining mechanism 120 is formed by two clamping elements 122 axially extending from a base structure 124 that includes the mounting member 112. The clamping elements 122 are extending from the base structure 124 into the lumen 108 and reaching to the proximal opening 106 or at the vicinity thereof. The clamping elements 122 are disposed on opposite sides of the coil engaging element 110 and are configured to be in the first state, in which they embrace or clamped to the external walls of a lead to thereby hold it in position so it cannot move with respect to the coil engaging element 110. In the second state, the clamping elements are more distanced from one another than the first state, thereby allowing a lead to move within said lumen with respect to the coil engaging element 110. The distance between the clamping elements 122 is adjusted by an adjuster 126 that is configured to control the distance between the clamping elements 122 to thereby switch between the first state and the second state. Each state is defined by a range of distances between the clamping elements 122, namely a range of clamping states, and the switching between the first and the second states is a threshold that depends on the size of the lead that is received in the device.

FIG. 1F is a cross-sectional view of the device 100 of the present disclosure and a part of a lead LD of a pacemaker or ICD to be inserted thereto and retained thereby, therefore blocking the movement of the coil of the lead for the extraction procedure. The lead LD has an external contour EC that fits in its dimensions into the lead receiving element 102 of the device 100. Thus, while moving the lead LD towards an insertion direction ID (as shown by the arrows), the coil engaging element 110 is inserted into the lead opening LO and engages the lead coil LC that resides within the lumen of the lead. The movement in the insertion direction ID causes the spring to be compressed until a portion pf the lead is inserted to the lumen 104 of the lead receiving element 102, and then the retaining mechanism 120 can switch between its states and retain the lead in position with respect to the coil engaging element 102, e.g. by tightly embrace the external contour EC of the lead LD.

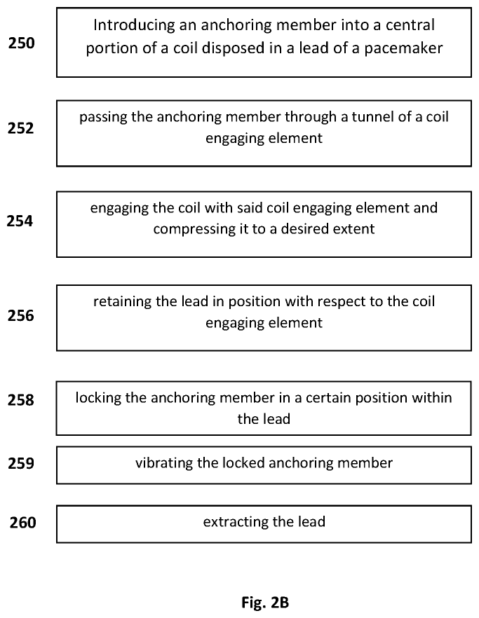

Reference is now being made to FIGS. 2A-2B, which are flow diagrams of different embodiments exemplifying a method for extracting a lead of a pacemaker or ICD according to an aspect of the present disclosure.

The method exemplified in FIG. 2A includes introducing an anchoring member into a central portion of a coil disposed in a lead of a pacemaker or ICD 250. The central portion of the coil forms an introduction path for the anchoring member along the entire length of the lead. The anchoring member is positioned at a desired location within the lumen of the lead, typically at a location about the distal end thereof. The method further includes passing the anchoring member, or a thread linked thereto, through a tunnel of a coil engaging element 252. It is to be noted that the steps of the method may be interchangeable in their order, and in particular step 250 is interchangeable with step 252. The coil engaging element is configured to be used in the method for engaging the coil and compressing it to a desired extent 254. Once the coil is sufficiently compressed, the method further includes retaining the lead in position with respect to the coil engaging element 256. The method further includes locking the anchoring member in a certain position within the lead 258 and extracting the lead from the subject 260. The retention of the lead with respect to the coil engaging element allows to extract the lead with the anchoring member that is locked to the lead without unnecessary movement of the coil that may damage the extraction procedure.

FIG. 2B exemplifies another embodiment of the method that differs from that of FIG. 2A by including vibrating the locked anchoring member 259 prior to the extraction of the lead from the subject 260. By applying vibrations to the lead, attachments of the lead to tissues of the subject tend to detach and the extraction is performed without harming the subject.

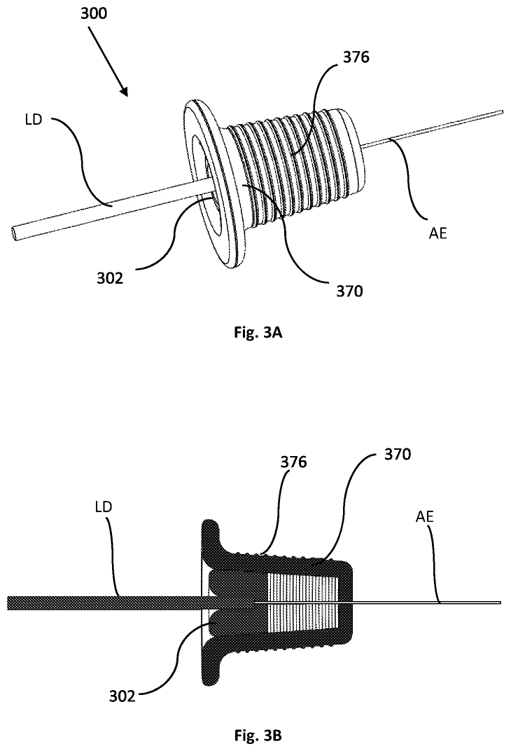

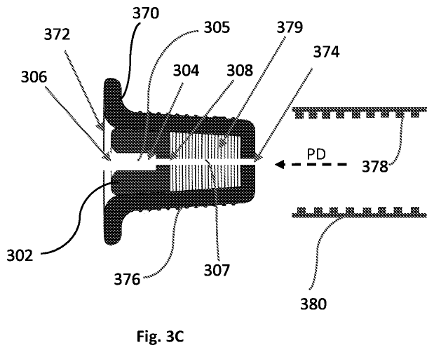

In the figures throughout the application, like elements of different figures were given similar reference numerals shifted by the number of hundreds corresponding to the number of the respective figure. For example, element 302 in FIGS. 3A-2B serves the same function as element 102 in FIGS. 1A-1F.

FIGS. 3A-3C are schematic illustrations of different views of a non-limiting example of an embodiment of the device for retaining a lead of a pacemaker or ICD for allowing its extraction from the body according to an aspect of the present disclosure. In FIGS. 3A-3C the device is shown with the lead received through the proximal end and the thread of the anchoring element extending from the lead and exiting the distal end. In FIG. 3C the device is shown alone without the lead and the thread.

The device 300 comprising a lead receiving element 302 (best seen in FIGS. 3B-3C) that is embraced by an embracing member 370. The lead receiving element 302 has a lumen 304 defined between a proximal opening 306 and a distal opening 308. The lumen 304 is formed of two sections having two different cross-sectional dimensions. The first section 305 is defined adjacent to the proximal opening 306 and the second section 307 is defined adjacent to the distal opening 308, wherein the dimension of the cross-section changes in the transition between the first section 305 to the second section 307. The first section 305 is sized to allow the receiving of the lead LD thereinto and the second section 307 is sized so as to (i) block the passing of the lead therethrough and (ii) allow the passing of an anchoring element AE or a part thereof, such as a thread of a locking stylet. The embracing member 370 is formed with an embracing member proximal opening 372 that is larger than the proximal opening 306 of the lumen 304 of the lead receiving element 302, allowing the lead LD to be received in the lead receiving element 302; and an embracing member distal opening 374 that is sized to allow the anchoring element AE or a part thereof to pass therethrough. The lead receiving element 302 is made of a pliable material that can be compressed in order to press a lead received in its lumen to retain it in position. The embracing member is deformable such that a deformation thereof causes the lead receiving element to press the lead. A portion of the external surface of the embracing member 370 is an external thread 376 that is configured to receive an internal thread 378 of a deformation-inducing element 380 such that as the threading operation is carried out towards the proximal direction PD, namely from the embracing member distal opening 374 side towards the embracing member proximal opening side, the embracing member is gradually pressed more and more on the lead receiving element 302, resulting in a gradually increase of pressure applied by the lead receiving element 302 on the lead received in its lumen 304 and the retention thereof in place. This occurs since the embracing member 370 has an internal lumen 379, in which the lead receiving element 302 is disposed, and the internal lumen size is gradually reducing along the proximal direction PD. In other words, the cross section of the internal lumen 379 of the embracing member 370 decreases in the progression along the proximal direction PD. The deformation-inducing element 380 has a constant cross section along its central axis that is smaller than the maximal cross section of the internal lumen 379 embracing member 370, therefore as the deformation-inducing element progress by being threaded in the proximal direction PD, the deformation of the embracing member 370 gradually increases, resulting in an increase of pressure applied on the lead received in the lumen of the lead receiving element 302.

1. A device for retaining a lead of a pacemaker or ICD for allowing its extraction from the body, comprising:
a lead receiving element having a lumen extending along an axis between a proximal opening and a distal opening of said lead receiving element, said proximal opening is shaped and sized to receive said lead, said distal opening is shaped and sized for (i) blocking passing of the lead and (ii) allowing passing of a part of a lead anchoring element anchored to said lead;
wherein at least a portion of the lead receiving element is deformable upon external application of force thereon, resulting in at least one of (1) a reduction of at least a portion of the lumen cross section and/or (2) an increase of pressure on a lead received in the lumen, thereby constituting a retaining mechanism configured to selectively switch between (i) a retaining state, in which it retains said lead in position within the lumen, and (ii) a non-retaining state, in which it allows movement of said lead within the lumen.

2. The device of claim 1, further comprising a coil engaging element defined between a first end and a second end, fixed with respect to the lumen, the first end is configured to engage a coil of said lead, wherein the coil engaging element is formed with a tunnel between the first end and the second end, said tunnel is shaped and sized for allowing passing of a part of a lead anchoring element anchored to said lead.

3. A device for retaining a lead of a pacemaker or ICD for allowing its extraction from the body, comprising:
a lead receiving element having a lumen extending along an axis between a proximal opening and a distal opening of said lead receiving element, said proximal opening is shaped and sized to receive said lead;
a coil engaging element defined between a first end and a second end, fixed with respect to the lumen, the first end is configured to engage a coil of said lead, wherein the coil engaging element is formed with a tunnel between the first end and the second end, said tunnel is shaped and sized for allowing passing of a thread of a lead anchoring element therethrough; and
a retaining mechanism configured to selectively switch between (i) a retaining state, in which it retains said lead in position with respect to the coil engaging element, and (ii) a non-retaining state, in which it allows movement of said lead with respect to the coil engaging element.

4. The device of claim 3, wherein:
the retaining mechanism is within said lumen between the lead receiving element and the coil engaging element; and
the first end of the coil engaging element extends over said proximal opening.

5. (canceled)

6. The device of claim 3, wherein:
the retaining mechanism comprises two or more clamping members that reside within the lumen and defining together a clamping state;
the clamping members are adjustable to allow a first range of clamping states defining said retaining state and a second range of clamping state defining said non-retaining state;
the device further comprises an adjuster configured to controllably adjust the clamping state of the clamping members; and
the coil engaging element extends in a space defined between the clamping members.

7. (canceled)

8. (canceled)

9. The device of claim 2, wherein the coil engaging element extending along the entire lumen and projecting through said proximal and distal openings.

10. (canceled)

11. The device of claim 2, further comprising a mounting member configured for mounting the coil engaging element within the lumen, wherein:
the mounting member is fixed to or integral with a part of the retaining mechanism; and
the mounting member is fixed to or integral with a part of the retaining mechanism.

12.-15. (canceled)

16. The device of claim 3, wherein at least a portion of the lead receiving element is deformable to decrease the lumen dimension and/or allowing to embrace said lead when it is received in the lumen, thereby constituting the retaining mechanism.

17. The device of claim 16, wherein the retaining mechanism is constituted by a deformation capability of at least a portion of the lead receiving element allowing to decrease the lumen volume and/or allowing to embrace said lead when it is received in the lumen.

18. (canceled)

19. The device of claim 1, wherein:
the retaining mechanism comprises an embracing member embracing at least a portion of said lead receiving element and is capable of selectively adjustment along a range of embracing pressures on said lead receiving element, between a minimal embracing pressure and a maximal embracing pressure, thereby resulting in the switch between said non-retaining state and said retaining state, and
said embracing member is formed with an embracing member proximal opening for allowing the passage of the lead therethrough, and an embracing member distal opening to allow the passage of the thread linked to the lead therethrough.

20. The device of claim 19, wherein said embracing member is deformable so as to allow said adjustment along a range of embracing pressures.

21. The device of claim 20, wherein the embracing member is formed with an external thread on its external surface for allowing an internal thread of a deformation-inducing element to be threaded thereto, thereby resulting in the deformation of the lead receiving element.

22. The device of claim 20, wherein at least a portion of said embracing member has a cross section profile that reduces along a central axis thereof from a proximal end towards a distal end, and wherein the deformation-inducing element has a constant cross section dimension along a central axis thereof, thereby the position of the deformation-inducing element over the external surface defines the amount of pressure applied in retaining said lead.

23. A kit comprising the device of claim 21 and said deformation-inducing element.

24. (canceled)

25. A method for extracting a lead of a pacemaker or ICD, comprising:
introducing an anchoring member into a central lumen of a coil disposed in said lead;
anchoring the anchoring member in a certain position within the lead;
passing a thread of the anchoring member from a proximal end of a lumen of a lead receiving element to a distal end of said lumen receiving element;
receiving a portion of said lead in said lumen of the lead receiving element;
deforming said lead receiving element for retaining the lead in position with respect to the lumen; and
extracting the lead by applying extracting force profile on said thread.

26. The method of claim 25, wherein said lead receiving element is embraced by an embracing member and the method comprising deforming at least a portion of said embracing member, resulting in said retaining.

27. The method of claim 26, wherein said deforming comprises threading an internal thread of a deformation-inducing element over an external thread of said embracing member, thereby resulting in said deforming and said retaining.

28. The method of any one of claim 24, comprising vibrating the locked anchoring member prior to said extracting.

29. A device according to claim 1 for use in a method for extracting a lead of a pacemaker or ICD.

30. The device for use of claim 29, wherein the method comprises:
introducing an anchoring member into a central lumen of a coil disposed in said lead;
anchoring the anchoring member in a certain position within the lead;
passing a thread of the anchoring member from a proximal end of a lumen of a lead receiving element to a distal end of said lumen receiving element;
receiving a portion of said lead in said lumen of the lead receiving element;
deforming said lead receiving element for retaining the lead in position with respect to the lumen; and
extracting the lead by applying extracting force profile on said thread.
